(19) United States
(12) Patent Application Publication
(10) Pub. No.: US 2020/0299777 A1
(43) Pub. Date: Sep. 24, 2020

(54) Methods for Resensitizing p53-Null Cells to Cancer Chemotherapy

(71) Applicant: UNIVERSITY OF SOUTH CAROLINA, COLUMBIA, SC (US)

(72) Inventors: PHILLIP BUCKHAULTS, BLYTHEWOOD, SC (US); CHANGLONG LIU, COLUMBIA, SC (US); CAROLYN E. BANISTER, COLUMBIA, SC (US)

(21) Appl. No.: 16/748,968

(22) Filed: Jan. 22, 2020

Related U.S. Application Data
(60) Provisional application No. 62/822,102, filed on Mar. 22, 2019.
(60) Provisional application No. 62/927,733, filed on Oct. 30, 2019.

(51) Int. Cl. C12Q 1/6886; A61K 31/711; C12N 15/86; C12Q 1/6869

(52) U.S. Cl. CPC C12Q 1/6886; A61K 31/711; A61K 45/06; C12Q 1/6869; C12N 2310/141; C12N 15/86

(57) ABSTRACT

The present disclosure is directed to methods for treating patients who have been diagnosed with cancers having a TP53 mutation. Aspects of the disclosure can be implemented to determine a treatment course for patients who have been diagnosed with a cancer having a TP53 mutation by excluding pharmaceutical compounds based, at least in part, on a genetic profile of the cancer. Additionally, aspects of the disclosure can be implemented to mitigate the effects of TP53 mutations by targeting biological pathways such as the spindle assembly checkpoint (SAC) to enhance or otherwise improve the efficacy of certain FDA-approved compounds. For instance, an example implementation of the disclosure can include a method for treating a patient who has been diagnosed with a cancer having a TP53 mutation. Advantages of the embodiments disclosed herein can provide patients with improved treatment efficacy when using chemotherapies or by reducing exposure to chemotherapeutics that demonstrate lower efficacy based on the genetic profile of the cancer.

### CROSS REFERENCE TO RELATED APPLICATION

This application claims filing benefit of U.S. Provisional Patent Application Ser. No. 62/822,102, having a filing date of Mar. 22, 2019, entitled “Spindle Assembly Checkpoint Inhibition Can Resensitize p51-Null Stem Cells to Cancer Chemotherapy;” and of U.S. Provisional Patent Application Ser. No. 62/927,733, having a filing date of Oct. 30, 2019, entitled “Spindle Assembly Checkpoint Inhibition Can Resensitize p51-Null Stem Cells to Cancer Chemotherapy,” both of which are incorporated herein by reference for all purposes.

### STATEMENT AS TO RIGHTS TO INVENTIONS MADE UNDER FEDERALLY SPONSORED RESEARCH

This invention was made with Government support under Contract No. U01 CA158428, awarded by the National Institutes of Health (NIH). The Government has certain rights in the invention.

### BACKGROUND

Chemotherapy resistance is a major problem in the clinical management of cancer patients. Drug resistance may arise due to intrinsic cellular resistance that is mediated through ATP-dependent membrane transporters or nuclear receptors by inhibiting drug accumulation or stimulating drug metabolism and inactivation. Inactivation of TP53 (also known as p53 or tumor protein) has been shown to result in resistance to chemotherapeutic drugs by abrogating p53-dependent apoptosis. p53 can prevent chromosomal instability through its ability to eliminate cells that are at risk of aberrant mitoses. Some studies suggest that p53-deficient cells are better at tolerating genetic stress produced by aberrant gene dosage. Hence, the absence of p53 can allow both the accumulation and survival of aneuploid cells.

Aneuploidy is a common characteristic of most cancer cells and has been suggested as a contributor to tumorigenesis. It has been reported that PLK1, a mitotic kinase, as a resistance mediator whose genomic, as well as pharmacological, inhibition restored drug sensitivity to trastuzumab emtansine (T-DM1) in HER2-positive breast cancer. The T-DM1 sensitization upon PLK1 inhibition was initiated by a spindle assembly checkpoint (SAC)-dependent mitotic arrest, leading to caspase activation, followed by DNA damage through CDK1-dependent phosphorylation and inactivation of Bcl-2/xl. Interestingly, up-regulation of PLK1 control the G2/M transition in the colorectal cancer RKO cells whose TP53 genes were inactivated and p53 inactive RKO cells were highly sensitive to PLK1 inhibitors. Additionally, missegregation of large numbers of chromosomes due to complete inactivation of the mitotic checkpoint results in cell death in human cancer cells.

What is needed in the art are methods for improving sensitivity of patients to chemotherapies. For instance, methods to resensitize p-53 deficient cells to cancer chemotherapies would be highly beneficial.

### SUMMARY

The present disclosure is directed in one embodiment to methods for treating patients having been diagnosed with cancers having a TP53 mutation. Aspects of the disclosure can be implemented to determine a treatment course for patients having been diagnosed with a cancer having a TP53 mutation by excluding pharmaceutical compounds. The method can be based, at least in part, on a genetic profile of the cancer. Additionally, aspects of the disclosure can be implemented to mitigate the effects of TP53 mutations by targeting biological pathways, such as the spindle assembly checkpoint (SAC), to enhance or otherwise improve the efficacy of certain FDA approved compounds. For instance, an example implementation of the disclosure can include a method for treating a patient who has been diagnosed with a cancer having a TP53 mutation. Advantages of the embodiments disclosed herein can provide patients with improved treatment efficacy when using chemotherapies or by reducing exposure to chemotherapeutics that demonstrate lower efficacy based on the genetic profile of the cancer.

### BRIEF DESCRIPTION OF THE FIGURES

A full and enabling disclosure of the present invention, including the best mode thereof to one skilled in the art, is set forth more particularly in the remainder of the specification, which includes reference to the accompanying figures, in which:

FIG. 1A illustrates results of generation of a TP53 knockout human embryonic stem cell line with a CRISPR-Cas9 gene editing system including the TP53 sgRNAs location (top left); Western blotting result showing p53 expression was abolished by TP53 knockout (bottom left); and representative images of morphology of TP53-KO and TP53-WT human H9 ES cells (right), scale bar: 1000 um.

FIG. 1B presents representative images for immunostaining of TP53-KO and TP53-WT human ESCs pluripotent markers Oct4, Sox2, and Nanog. Nuclei are visualized with Hoechst staining, scale bar: 200 um.

FIG. 1C presents cell proliferation of TP53-KO and TP53-WT human ESCs as determined by resazurin at different day as indicated. **, p<0.01.

FIG. 1D presents results of a functional p53 test for TP53-KO human ESCs using Nutlin-3a that could accumulate p53 in cells by inhibiting MDM2 interaction with p53 protein.

FIG. 2A illustrates a heat map of area under curve (AUC) response in a systematic screen of FDA approved drugs for wild type (WT) and knockout (KO) cells.

FIG. 2B illustrates a scatter plot of drugs screening. Left panel: A volcano plot representation of student's t-test results shows the magnitude (The log 10 of ratio between AUC of TP53-WT and of TP53-KO, x-axis) and significance (p value, y-axis) of all drug-TP53 associations. Each dot represents a single drug and red dots indicate the drugs were statistical significance between AUC of TP53-WT and TP53-KO, p value<0.01. Right: Scatter plot is magnified views of p53-null hESCs resistant drugs and the drug names are showing.

FIG. 2C illustrates the panel of 27 p53-null hESCs resistant drugs classified according to their therapeutic targets. (A single drug may be included in multiple categories.)

FIG. 3A presents a volcano plot representation of student's t-test results on AUC showing the magnitude and significance of all drug-TP53 associations. The name of common drugs for colorectal cancer and epithelial ovarian cancer chemotherapy are shown.

FIG. 3B presents the viability of TP53-WT and TP53-KO hESCs after Nutlin-3a treatment as a positive control.

FIG. 3C presents the viability of TP53-WT and TP53-KO hESCs after Docetaxel treatment.

FIG. 3D presents the viability of TP53-WT and TP53-KO hESCs after Paclitaxel treatment.

FIG. 3E presents the viability of TP53-WT and TP53-KO hESCs after Olaparib treatment.

FIG. 3F presents the viability of TP53-WT and TP53-KO hESCs after Carboplatin treatment.

FIG. 3G presents the viability of TP53-WT and TP53-KO hESCs after Cisplatin treatment.

FIG. 3H presents the viability of TP53-WT and TP53-KO hESCs after Capecitabine treatment.

FIG. 3I presents the viability of TP53-WT and TP53-KO hESCs after Fluorouracil treatment.

FIG. 3J presents the viability of TP53-WT and TP53-KO hESCs after Irinotecan treatment.

FIG. 3K presents the viability of TP53-WT and TP53-KO hESCs after Oxaliplatin treatment.

FIG. 3L presents the viability of TP53-WT and TP53-KO hESCs after Trifluridine treatment. In all of FIG. 3B-3L, the p-values from two-tailed student's t test on the AUC are shown on the plots.

FIG. 4A illustrates a workflow diagram of genome-scale CRISPR screening on TP53-KO hESCs with drugs.

FIG. 4B illustrates a Boxplot of CRISPR gene scores of 927 non-essential genes and 1,580 essential fitness genes in Cisplatin and DMF treatment. P values between gene CRISPR score of essential genes and nonessential genes were from student's t test.

FIG. 4C illustrates gene sets enrichment analysis of essential biological process of cell proliferation in DMF treatment TP53-KO hESCs and Cisplatin treatment TP53-KO hESCs.

FIG. 5A illustrates a graph displaying gene rank ordered by negative selection score from MAGeCK output from Cisplatin treatment versus DMF. Significant genes between Cisplatin treatment and DMF control (p<0.01) from MAGeCK analysis and genes involving in spindle assembly checkpoint and chromosome organization were identified.

FIG. 5B illustrates a scatterplot of CRISPR gene scores of Cisplatin treated TP53-KO hESCs and DMF treated TP53-KO hESCs. Light dots: all genes. Dark dots: the 137 significant genes between Cisplatin treatment and DMF treatment from MAGeCK software.

FIG. 5C illustrates the position of 6 sgRNAs targeting gene ZNF207/BuGZ or BRD7 shown in ordered rank of all library sgRNA counts. Dark lines: sgRNAs targeting the same gene.

FIG. 5D illustrates the top eight GO biological processes of GO analysis for the 137 significant genes.

FIG. 5E presents results following infection of Cas9-expressed TP53-KO human ESCs with ZNF207/BuGZ sgRNAs or control OR1C1 sgRNAs. Following, the ESC's were treated with DMF and Cisplatin, respectively. The ZNF207/BuGZ or OR1C1 knockout cells were monitored by qPCR. *, p<0.05. Kaplan-Meier survival plot was generated from the cohort of ovarian cancer patients according to ZNF207 expression level. P values on the plots are from log-rank test for the comparisons of the low and high ZNF207 expression groups.

FIG. 5F illustrates results of 449 patients that were deemed TP53-mutated and received chemotherapy that contained platin.

FIG. 5G illustrates results of 82 patients that were deemed TP53-wt and received chemotherapy that contained platin.

FIG. 6A illustrates a 3-dimensional plot displaying synergy score versus varying concentrations of cisplatin and paclitaxel from three 96-well plates on TP53-KO hESCs.

FIG. 6B illustrates a 3-dimensional plot displaying synergy score versus varying concentrations of cisplatin and paclitaxel from three 96-well plates on RKO colon cancer cells TP53-KO-A.

FIG. 6C illustrates a 3-dimensional plot displaying synergy score versus varying concentrations of cisplatin and paclitaxel from three 96-well plates on RKO colon cancer cells TP53-KO-B.

FIG. 6D illustrates a predicted model that Cisplatin causes cell death with dysregulation of chromosome segregation. Moderate levels of genetic instability, caused by mutations in mismatch repair genes or by missegregation of one to three chromosomes per division, promote cell growth and tumorigenesis (top panel). High levels of genetic instability, caused by chemotherapeutic agents such as Cisplatin or missegregation of large numbers of chromosomes per division, result in cell death and tumor suppression (bottom panel).

Repeat use of reference characters in the present specification and drawings is intended to represent the same or analogous features or elements of the present invention.

### DETAILED DESCRIPTION

Reference now will be made to embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only and is not intended as limiting the broader aspects of the present invention, which broader aspects are embodied exemplary constructions.

The present disclosure is directed to methods for treating patients having been diagnosed with cancers having a TP53 mutation. Aspects of the disclosure can be implemented to determine a treatment course for patients having been diagnosed with cancer by excluding pharmaceutical compounds based at least in part on a pathology of the cancer. Additionally, aspects of the disclosure can be implemented to mitigate the effects of TP53 mutations by targeting biological pathways such as the spindle assembly checkpoint (SAC) to enhance or otherwise improve the efficacy of certain FDA approved compounds.

Some implementations of the disclosure can include methods for treating a patient who has been diagnosed with a cancer having a TP53 mutation. Generally, methods for treating the patient, in accordance with the disclosure, include delivering an inhibitor to the patient via an administration route. As used herein, the inhibitor can be used to target an aberrant gene or a protein derived from an aberrant gene. In some implementations, the aberrant gene can be associated with a biological pathway (e.g., spindle cell assembly checkpoint regulation). Alternatively, the aberrant gene may include one or more genes from various pathways. Several non-limiting examples of genes associated with the spindle cell assembly checkpoint (SAC) pathway that can be used in implementations of the disclosure include: ZNF207, BRD7, PCID2, CDK9, MAD2L2, KDM1A, PUM2, GATA2 and TRIP1. Example aberrant genes are included herein in Table 2, which includes 137 genes.

For implementations of the disclosure, delivering the inhibitor can include delivering one or more compounds that reduce gene expression (e.g., by inhibiting transcription, translation, or other cellular processes) or interfere with protein function (e.g., by binding to a region of the protein) of one or more aberrant genes. In an example implementation, reducing gene expression can be accomplished by inducing a controlled cellular mutation, such as by using a CRISPER/Cas9 cassette. Additionally, or alternatively, reducing gene expression can utilize delivery of micro-RNA (miRNA) to reduce translation of RNA derived from the genes through selective binding.

In some instances, the miRNA can include a substantially complementary sequence to a transcription product of one of the aberrant genes. As an example, miR-216a-5p displays a pairing region with a portion of ZNF207 3′ untranslated region. For implementations of the disclosure, the substantially complementary sequence can include the complementary sequence or a modified complementary sequence. The modified complementary sequence can include one or more additions, deletions, or substitutions to modify the complementary sequence without reducing the ability to bind and inhibit the miR sequence. The number of modifications that still result in inhibition can be determined using an analytical technique including, but not limited to, a circular dichroism (CD) spectrometry and/or calorimetry.

Additionally, or alternatively, delivering the inhibitor can include delivering one or more compounds that reduce the function of a translation product (e.g., a protein) of the aberrant gene. In an example implementation, the inhibitor can include the binding region of an antibody such as a variable region present on either the heavy chain, light chain, or both. In another example implementation, the inhibitor can include the complete antibody. Antibodies that can be used as inhibitors, in accordance with this disclosure, may include monoclonal systems that target a single epitope of the protein or polyclonal systems that target multiple epitopes on one or more proteins produced as translation products from the aberrant genes.

In certain implementations, delivering the miRNA can include delivering a vector, including heterologous DNA expressing the miRNA. In this manner, a vector targeting a specific cancer cell, such as a breast cancer cell, may be used to direct the treatment to a certain cancer and/or an intracellular environment which may provide an advantage for certain treatments. A quantity of miRNA oligonucleotides can be generated from this and similar expression systems. Recombinant expression can be usefully accomplished using a vector, such as a plasmid. The vector can include a promoter operably linked to a sequence encoding the miRNA. The vector can also include other elements required for transcription and translation. As used herein, vector refers to any carrier containing exogenous DNA. Thus, vectors can generally refer to agents that transport the exogenous nucleic acid into a cell and can include a promoter yielding expression of the nucleic acid in the cells into which it is delivered. Vectors include, but are not limited to, plasm ids, viral nucleic acids, viruses, phage nucleic acids, phages, cosmids, and artificial chromosomes. A variety of prokaryotic and eukaryotic expression vectors suitable for carrying, encoding and/or expressing the miRNA are encompassed herein. Such expression vectors include, for example, pET, pET3d, pCR2.1, pBAD, pUC, and yeast vectors.

A variety of regulatory elements can be included in an expression cassette and/or expression vector, including promoters, enhancers, translational initiation sequences, transcription termination sequences, and other elements.

For embodiments of the disclosure that include inducing a controlled cellular mutation, generally, these methods include delivering a vector including heterologous DNA expressing one or more sgRNAs. Example vectors for use with these methods, in accordance with the disclosure, can include lentiviruses such as HIV that have been modified to include the heterologous DNA.

In combination with delivering the inhibitor, certain methods in accordance with the disclosure can also include delivering a therapeutic agent. Example therapeutic agents that can be used in accordance with the disclosure include pharmaceutical compounds such as chemotherapeutics. An example advantage of embodiments of the disclosure is that these compounds generally do not display efficacy in cancers having a TP53 mutation. However, providing an inhibitor to the patient, according to example embodiments of the disclosure, can be used to resensitize the cancer which may in turn improve treatment efficacy and/or patient outcome. Thus, certain implementations can provide an advantage to patients and healthcare providers by diversifying treatment courses that can be used to reduce or eliminate the cancer.

Several non-limiting examples of the therapeutic agent include: Bendamustine hydrochloride, Bleomycin sulfate, Carboplatin, Carmustine, Chlorambucil, Cisplatin, Cladribine, Clofarabine, Cytarabine hydrochloride, Decitabine, Dexrazoxane, Estramustine phosphate sodium, Etoposide, Irinotecan hydrochloride, Melphalan hydrochloride, Mitomycin, Olaparib, Osimertinib, Oxaliplatin, Pipobroman, Teniposide, Thiotepa, Topotecan hydrochloride, Triethylenemelamine, Trifluridine, Uracil mustard, and Valrubicin. For patients not undergoing treatment with an inhibitor as disclosed herein, additional implementations of the disclosure may include directing physicians or other healthcare workers to exclude these therapeutic agents from a cancer treatment course.

For example, another implementation of the disclosure can include a method for selecting a treatment course for a patient having been diagnosed with cancer. For methods directed to selecting a treatment course, generally these include determining a genetic profile for the cancer, where the genetic profile includes at least the TP53 genetic sequence for one allele of the gene. These methods can also include comparing the TP53 gene sequence determined for the patient to a native (wild type) TP53 gene sequence. For implementations of the disclosure, this comparison should be made using the same species. As an example, if the cancer is in a human patient, the native TP53 sequence should include the human TP53 gene sequence. Based at least in part on the comparison, a medical professional, such as a doctor, can then determine a treatment course that excludes (i.e., does not include) one or more resistant drugs.

As used herein, the resistant drug can include one or more of the therapeutic agents disclosed herein (e.g., Cisplatin).

In some instances, comparing the TP53 gene sequence for the patient to the native TP53 gene sequence can demonstrate a mutation. For certain implementations, selecting the treatment course can be based in part on the mutation including a deletion of at least a portion of the TP53 gene sequence (compared to the native TP53 gene sequence). In some implementations, the portion of the TP53 gene sequence can include the entire TP53 gene sequence.

Methods for selecting a treatment course in accordance with this disclosure may also include obtaining a biopsy of the cancer. For these implementations, determining the genetic profile can include sequencing DNA from cells obtained from the biopsy to determine a genetic sequence for at least one allele encoding the TP53 gene.

### Example 1

Example 1 discusses various methods and provides exemplary embodiments that may be understood in conjunction with the Drawings and Description provided herein. The materials and conditions described in the example are demonstrative and are not meant to constrain the scope of the disclosure only to the materials and conditions used.

### Materials and Methods

### Cell Lines Culture

Human H9 embryonic stem cells (ESCs) (Lot No.: WIC-WA09-MB-001, WiCell, Wisconsin) and derivatives were maintained at 37° C., 5% CO₂ in chemical-defined medium TeSR-E8 medium (Stemcell Tech.) with 100 U/ml penicillin & 100 μg/ml streptomycin (Gibco) on Matrigel-coated (# CB40230A, Corning) tissue culture vessels. Authentication of H9 ESCs were performed by WiCell. ESCs were passaged every 4 to 6 days to maintain sub-confluence using 0.5 mM EDTA as described previously. Human colon cancer RKO cells (kindly given by Dr. Bert Vogelstein) and its derivatives were maintained at 37° C., 5% CO₂ in McCoy's 5A media (Fisher) supplemented with 10% FBS and 100 U/ml penicillin & 100 μg/ml streptomycin (Gibco). RKO cells were passaged every 3 to 4 days to maintain sub-confluence (authentication of RKO cell line was performed by JHU-GRCF Biorepository & Cell Center). Cells were screened for mycoplasma before experiments using a MycoAlert™ Mycoplasma Detection Kit (Lonza).

All cell lines were passaged in the laboratory for no more than 30 passages after resuscitation.

TP53 Knock Out in Human Embryonic Cells and RKO Cells with CRISPR/Cas9

TP53 knockout hESCs and RKO cells were generated using CRISPR/Cas9 as described previously with minor modifications. Briefly, human codon-optimized Streptococcus pyogenes wild type Cas9 (Cas9-2A-GFP) was obtained from Addgene (#44719). Chimeric guide RNA expression cassettes with different small guide RNA, TP53_Up_sgRNA: 5′-CCATTGTTCAATATCGTCCG-3′ (SEQ ID NO: 1) and TP53_Down_sgRNA: 5′-GGGCAGCTACGGTTTCCGTC-3′ (SEQ ID NO: 2) were ordered as gBlock. These gBlocks were amplified by PCR using primers: gBlock_Amplifying_F: 5′-TGTACAAAAAAGCAGGCTTTAAAGG-3′ (SEQ ID NO: 3) and gBlock_Amplifying_R: 5′-TAATGCCAACTTTGTACAAGAAAGC-3′ (SEQ ID NO: 4). The PCR product was purified by Agencourt Ampure XP PCR Purification beads according to manufacturer's protocol (Beckman Coulter). 1.5 μg of Cas9 plasmid and 0.5 μg of each gRNAgBlock were co-transfected into hESCs via Lipofectamine™ 3000 (Thermo Fisher Scientific). For TP53-KO hESCs, the transfected cells were cultured in TeSR-E8 medium with 1 μM Nutlin-3a for one week. For TP53-KO RKO cells, single clones were picked up and validated by PCR and Western blotting.

### Cell Viability Assay

NCI Approved Oncology Drug Set IV containing 127 FDA-approved anticancer drugs was obtained from the NCI under a material transfer agreement. TP53 knockout or wild-type human ES cells were seeded in 96-well microplates in E8 medium, with 6,000 cells per well that would reach about 85% confluent at the end of the assay. Human ES cells were plated one day before treatment with a 7-point two-fold dilution series (starting with 10 μM) of each compound or solvent (dimethyl sulfoxide, DMSO or Dimethylformamide, DMF) control. After 72 hours incubation, cells were stained with 35 μM resazurin (Sigma), then quantification of fluorescent signal intensity was performed on Thermo Fluorskan™ Ascent plate reader at excitation and emission wavelengths of 544/590 nm. Data were normalized to the solvent control (DMSO or DMF) group. The area under a curve (AUC) was calculated using auc function (flux package) in R. The two-side t-test was performed with tlest function in R. The hierarchical clustering analysis of drugs AUC pattern in different samples was carried out using heatmap.2 function (gplots package) in R (All R code available). Viability response curves of Cisplatin and Paclitaxel on TP53-KO hESCs or RKO cells were generated using drc package (Analysis of Dose-Response Curves) in R.

### Lentiviral CRISPR Library Amplification and Cell Transduction

The human GeCKO lentiviral library lentiCRISPRv2 in one plasmid was bought from Addgene (cat #1000000048) as library A and library B. The library was amplified in accordance to the author's recommendations. Briefly, 2 μL of the 50 ng/μL lentiCRISPRv2 plasmid was electroporated with 25 μl of the Lucigen Endura™ electro competent cells (cat #60242) in 1.0 mm cuvette using a GenePulser Xcell™ (Bio-Rad) apparatus at the following settings: 10 ρF capacitance, 600Ω resistance, 1800 V. Then, transfer cells were placed in recovery medium to the final volume of 1 ml, and the above procedure was repeated for a total of 4 electroporations for each module of the lentiCRISPRv2. The recovered transformed bacteria in 4 ml medium were incubated at 250 rpm at 37° C. for 1 hour, and plated onto pre-warmed twenty 10 cm dishes with Ampicillin LB-agar for 14 hours at 32° C. The grown colonies were recovered from the plates by pipetting/scrapping in LB-broth. The plasmid DNA from transformed cells was purified using QIAfilter Plasmid Maxi Kit (Qiagen).

For lentiviral production, the day before transfection, 293T cells were seeded at 1.2×10⁷ cells per 150-cm² dish. On the next day, sixty micrograms plasmid DNA was used for transfection of one 150 mm dish. The DNA cocktail contained 10.5 μg envelope-coding plasmid pMD.G, 19.5 μg of the packaging plasmid pSPAX2, and 30 μg of transgene vector plasmid by CaCl₂) method according to procedure published. Next day, the culture medium was replaced, and cells were grown for another 48 hours. Supernatants from the twenty 150-mm dish with transfected 293T cells were harvested, combined, and clarified through a 0.45 μm cellulose acetate filter (Millipore, Cat. No: SCHVU01RE). Then, the virus supernatants concentrated using PEG6000 and concentrated virus were stored in −80° C. freezer.

### GeCKO CRISPR Library Screening

Viral transduction was carried out through spinfection with a MOI of 0.3 to assure that no more than one viral particle enters a given cell. The spinfection was conducted for 2 hours at 1000×g and 37° C. and then incubated overnight a 37° C. Cells were trypsinized and transferred to matrigel coated 150-mm culture dishes containing growth media plus 0.4 μM puromycin (Sigma) to select for successful transduction. After 3 days of selection, all remaining cells should be successfully transduced; these cells were then collected and plated. The lentiviral construct will insert a copy of the puromycin resistance, a single sgRNA, and Cas-9 genes into the cell DNA through retroviral activity, allowing the transduced cells to pass the resistance and CRISPR activity to all daughter cells. This protocol was conducted using 1.1×10⁸ starting cells to give −200-fold coverage of the library A and B, respectively. After selection, survived cells were divided into two parts. One part (>20 million) was treated with 200 nM Cisplatin. Another part (>20 million) was treated with control DMF. Cells were passaged after reaching 90% confluence. After about 14 doublings, cells were then collected for DNA extraction.

### DNA Extraction and Sequencing

DNA was extracted from cell trypsinate using Blood & Cell Culture DNA Midi Kit (Qiagen). The sgRNA sequences present in the collected DNA were amplified through PCR using primers that attach Illumina sequencing recognition sites and barcodes. A total of 100 μg of genomic DNA template was used per sample.

For each sample, 25 separate 100 μl reactions were performed with 4 μg genomic DNA in each reaction using KAPA Real-time Library Amplification Kit (KAPA Biosystems) and then combined the resulting amplicons. Primers sequences to amplify lentiCRISPR sgRNAs for PCR were:

Forward primer for PCR:
(SEQ ID NO: 5)
AATGATACGGCGACCACCGAGATCTACACTCTTTCCCTACACGACGCTCT
TCCGATCTNNNNNTCTTGTGGAAAGGACGAAACACCG.
(NNNNN: variable base sequence to introduce
diversity).
Reverse primer:
(SEQ ID NO: 6)
CAAGCAGAAGACGGCATACGAGATNNNNNNGTGACTGGAGTTCAGACGTG
TGCTCTTCCGATCTTGTGGGCGATGTGCGCTCTG.
(NNNNNN: Sample Barcode).
Sequencing primer:
(SEQ ID NO: 7)
ACACTCTTTCCCTACACGACGCTCTTCCGATCT

The PCR product was purified using the Agencourt AMPure XP bead bound purification kit. The purified PCR product was then sequenced on an Illumina HiSeq 2500.

### Data Processing and Analysis

The Illumina NextSeq raw FASTQ files were processed by MAGeCK software with default parameters using sgRNA sequence list for all genes from the GeCKO v2 library A and B to produce raw counts tables. The numbers of uniquely aligned reads for each library sequences were calculated. Then, the numbers of reads for each unique sgRNA for a given sample were normalized as following:

$$normalized reads per sgRNA = \frac{reads per sgRNA}{total reads for all sgRNA in sample} \times {10}^{6} + 1$$

sgRNA counts from GeCKO library A and B were merged after normalization.

For negative selection analysis, MAGeCK-RRA was used as the MAGeCK analysis pipeline. The output file with gene summary was used for downstream analysis. Gene ontology analysis for overrepresented genes was performed using R package clusterProfiler.

For each gene in each sample, its CRISPR score was defined as the average log 2 fold-change in the abundance of all single guide sgRNAs targeting the gene after 14 population doublings.

$$CRISPR gene score = average [ log 2 ( \frac{End sgRNA abundance}{Initial sgRNA abundance} ) ]$$

The cell-essential genes are involved in fundamental biological processes. Gene set enrichment analysis was performed on genes ranked by CRISPR gene score.

### Validation of Spindle Assembly Checkpoint Gene ZNF207/BuGZ

Human TP53 knockout embryonic stem cells were infected at low MOI by viruses produced using pCLIP-Cas9-Nuclease-EFS-Blast (TransOMIC) and selected using blastcidin (10 μg/ml). The stable Cas9-expressing TP53-KO hESCs were infected with viruses with two sgRNAs against gene ZNF207/BuGZ (# TEDH-1090944, TransOMIC) or control gene OR1C1 (# TEDH-1055091, TransOMIC). These two sgRNAs could induce fragment deletion. We treated these cells with either 200 nM Cisplatin or vehicle DMF. We used realtime quantitative PCR to quantify the ZNF207 knockout locus, using gRNA flanking primers that only amplify when the intervening sequence has been deleted.

ZNF207-KO-F1:
(SEQ ID NO: 8)
GGTTGGGAAAGTGAGGGATT
ZNF207-KO-R1:
(SEQ ID NO: 9)
AACACTTCTCACAGGAACTTGC
OR1C1-KO-F1:
(SEQ ID NO: 10)
CAGCCTCCTTCTGTGTGTGA
OR1C1-KO-R1:
(SEQ ID NO: 11)
TGCTTGCCCTGAGTAGAGGT

The total genomic DNA was monitored by LINE gene using LINE primer:

LINE-F:
(SEQ ID NO: 12)
AAAGCCGCTCAACTACATGG
LINE-R:
(SEQ ID NO: 13)
CTCTATTTCCTTCAGTTCTGCTC.

The relative percentage of ZNF207 or OR1C1 knockout cell number was defined as:

2^{-ΔCT(Gene-LINE)}/2^{-ΔCT(Gene-LINE)} at Day0.

### Drug Synergy Experiment and Analysis

A Paclitaxel and Cisplatin drug matrix (12×8) in a 96-well plate was made for drug synergy experiment. Paclitaxel on row was an 11-point two-fold dilution series with starting concentration of 0.0016 μM, and Cisplatin on column was a 7-point two-fold dilution series with starting concentration of 2 μM, 5000 TP53-KO hESCs per well or 2000 TP53-KO RKO cells per well were plated on three 96-well plates one day before treatment with Paclitaxel and Cisplatin drug matrix. After 72 hours incubation for TP53-KO hESCs and 96 hours incubation for TP53-KO RKO cells, cells were stained with 35 μM resazurin (Sigma), then quantification of fluorescent signal intensity was performed on Thermo Fluorskan™ Ascent plate reader at excitation and emission wavelengths of 544/590 nm. The drug synergy scores were evaluated using ZIP model (Zero Interaction Potency model) in synergyfinder package.

### Immunostaining and Microscopy

Cells were fixed in 4% paraformaldehyde in DPBS and incubated for 20 minutes at room temperature. After washing 3 times with DPBS, the cells were permeabilized and blocked with blocking buffer (0.1% Triton-X 100 and 10% FCS in DPBS) for 1 hour at room temperature, and then incubated with primary antibodies in blocking buffer. Anti-Oct4 (1:2000, cat #:561555, BD Pharmingen™), anti-Nanog (1:100, cat #: 560109, BD Pharmingen™) and anti-Sox2 (1:100, cat #: 561469, BD Pharmingen™) antibodies overnight at 4° C. Then they were incubated with secondary antibodies: anti-rabbit IgG, anti-mouse IgG or anti-mouse IgM conjugated with Alexa 488 (1:1000, cat #: A11004, Invitrogen) or Alexa 568 (1:1000, cat #: A10667, Invitrogen) in blocking buffer for 1 hour at room temperature. The cells were counterstained with 4,6-diamidino-2-phenylindole (DAPI) for 10 minutes. Images were taken using microscope equipped with monochrome EMCCD camera.

### Western Blotting Analyses

Cell lysates were prepared using RIPA buffer (150 mM NaCl, 1% Triton X-100, 0.5% sodium deoxycholate, 0.1% SDS and 50 mMTris-HCl, pH 8.0), supplemented with protease inhibitor cocktail (cat #: P8340, Sigma). The concentration of protein was determined using Pierce BCA Protein Assay Kit (cat #: 23227, Thermo Scientific). 20 ug of denatured cell lysates were separated by electrophoresis on 10% or/and 7% SDS-PAGE, and then were transferred to hydrophobic PVDF. The blot was blocked with TBST (10 mM Tris-HCl, pH 7.5, 150 mM NaCl, and 0.1% Tween-20) containing 5% non-fat dry milk followed by an overnight incubation with primary antibody in TBST at 4° C. overnight. After washing with TBST, the membrane was incubated with horseradish peroxidase (HRP)-conjugated secondary antibody for 1 hour at room temperature with constant agitation. Signals were raised with SuperSignal™ West Pico Chemiluminescent substrate (cat #: 34077, Thermo Scientific) and detected using a ChemiDoc™ MP imaging system (Bio-RAD). Primary antibodies for p53 (1:400, cat #: sc-126, Santa Cruz), β-actin (1:1,000, cat #: 4970, CST), and horseradish peroxidase-linked secondary antibodies for mouse IgG (1:2,000, cat #: 7076, CST) and rabbit IgG (1:2,000, cat #: 7074, CST) were used.

### Statistical Analyses

The data was evaluated by unpaired t-test (student t-test) using the GraphPad Prism software (GraphPad Software, Inc), and values of P<0.05 were considered to be significant (indicated by asterisks in figures). The error bars represent the standard deviation (S.D.).

### Results

### Generating and Characterizing TP53 Knockout Derivatives of Human ESCs.

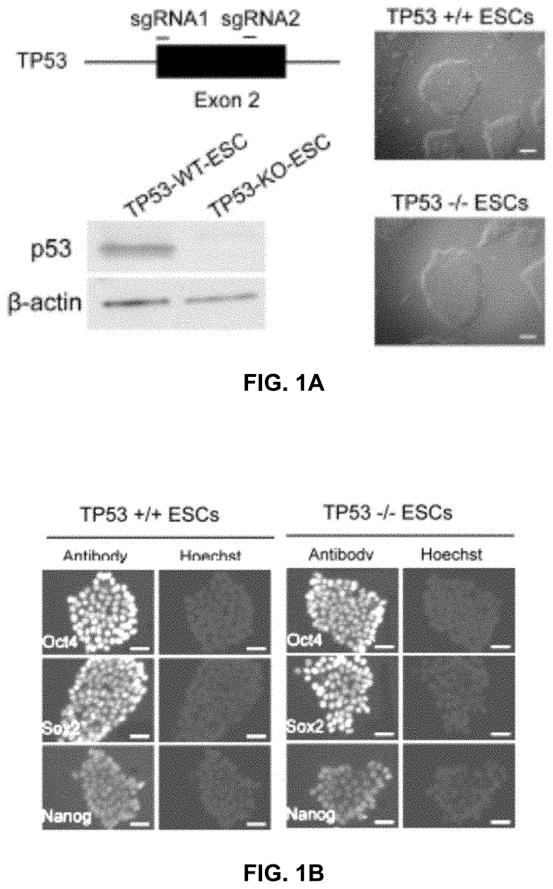

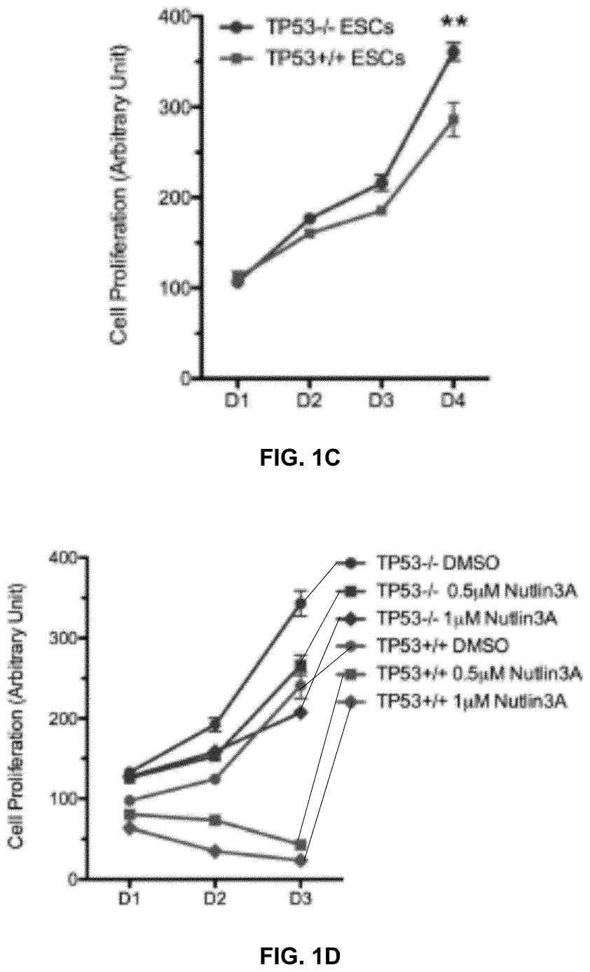

In order to screen for p53-dependent drug sensitivity, without confounding interactions from other gene mutations, the human embryonic stem cell line E9 (hESC) was chosen, which is wild-type for TP53 (TP53-WT) and has few known acquired gene mutations. Embryonic stem cells are also a reasonable model for cancer stem cell biology. TP53 knockout (TP53-KO) derivatives of hESCs were constructed using CRISPR/Cas9 genome editing, targeting two locations within exon 2 of the TP53 gene (FIG. 1A). gBocks encoding two small guide RNAs (sgRNA) were co-transfected with Cas9 vector into human ES cells, and TP53 defective cells were selected by growing the cells in the presence of 1 μM Nutlin-3a, which is an MDM2 inhibitor and potent activator of p53-induced arrest and apoptosis. TP53-KO hESCs retained morphology similar to their parental TP53 wild-type hESCs (FIG. 1A). TP53 gene locus of TP53-KO hESCs were confirmed by DNA sequencing, and the transcriptional level of TP53 was abolished in TP53-KO hESCs compared to TP53-WT hESCs. The p53 protein was visualized in the parental TP53-WT hESCs by Western blotting, and the absence of p53 protein was confirmed in TP53-KO hESCs (FIG. 1A). Furthermore, to determine if loss of p53 affected the pluripotency of embryonic stem cells, immuno-staining of TP53 WT and KO hESCs was performed for pluripotent markers OCT4, SOX2, and NANOG. Both the TP53-WT parental hESCs and the TP53-KO hESCs express these pluripotent stem cell markers (FIG. 1B) suggesting that TP53-KO hESCs were still phenotypically embryonic stem cells. We observed a modest increase in the rate of cell proliferation in the TP53-KO hESCs compared to TP53-WT hESCs (FIG. 1C). Finally, as expected, TP53-KO hESCs were resistant to the growth inhibitory effects of Nutlin-3a, whereas the TP53-WT hESCs were severely growth arrested at very low concentrations of Nutlin-3a (FIG. 1D).

### Loss of p53 Function Confers Resistance to Multiple Cancer Chemotherapy Drugs in Human Embryonic Stem Cells

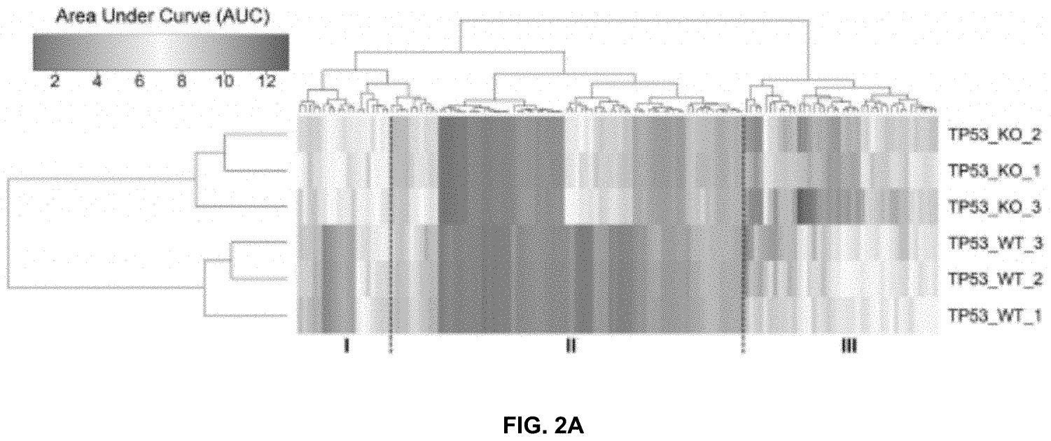

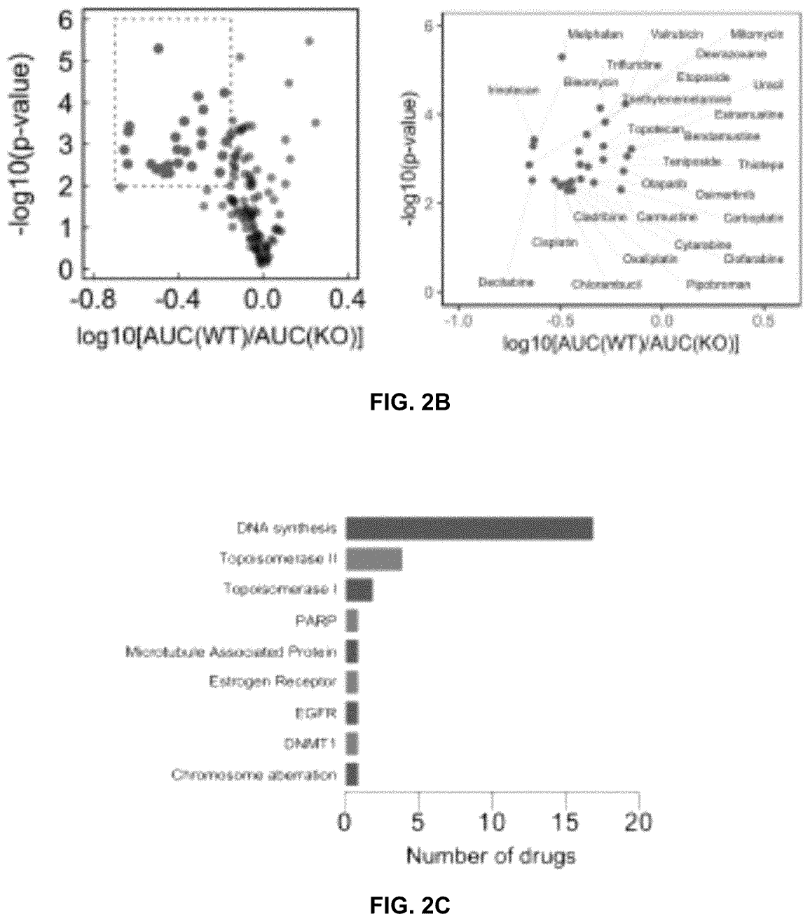

Clinical studies have demonstrated the prognostic relevance of mutated p53, often associating mutant TP53 with resistance to alkylating agents, anthracyclines, antimetabolites, anti-estrogens, and EGFR-inhibitors. To establish a cause and effect relationship between p53 inactivation and resistance to specific chemotherapies, the NCI Approved Oncology Drug Set IV was screened, which is a panel of 127 FDA-approved anticancer drugs against TP53-WT and TP53-KO hESCs and determined which drugs were less effective after mutational inactivation of p53. Dose-response measurements were performed in experimental triplicates, and the effects of 72 hours of drug treatment on hESC viability was measured using a fluorescent resazurin cell viability assay. The area under the curve (AUC) was used to quantify the sensitivity of each cell line to each drug. Unsupervised hierarchical clustering via the AUC measurements drove the 127 drugs into three distinct groups: (I) drugs for which TP53-KO hESCs are more resistant to TP53-KO hESCs than TP53-WT hESCs; (II) drugs for which both TP53-WT and TP53-KO hESCs are equally sensitive; and (III) drugs for which both TP53-WT and TP53-KO hESCs are resistant (FIG. 2A). It was found that 27 drugs were significantly different between TP53-WT and TP53-KO hESCs, and all the drugs were less effective on the TP53-KO derivatives than the TP53-WT parental hESCs (FIG. 2B). These 27 p53-null hESCs resistant drugs can be classified according to their therapeutic targets and most of these drugs inhibit DNA synthesis and/or topoisomerase (FIG. 2C).

### TP53 Loss Causes Resistance to Irinotecan, Oxaliplatin, Cisplatin and Olaparib

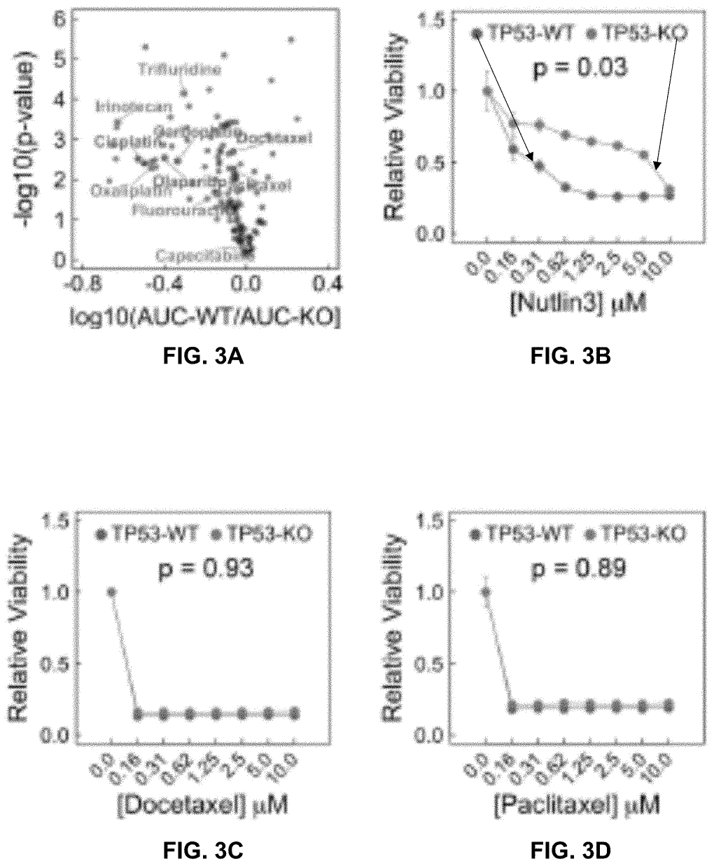

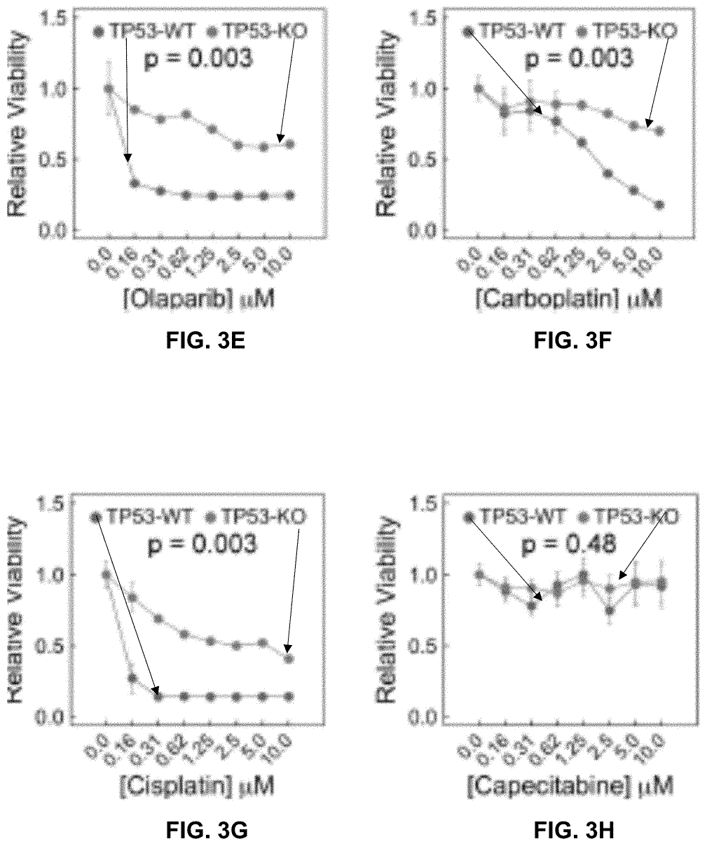

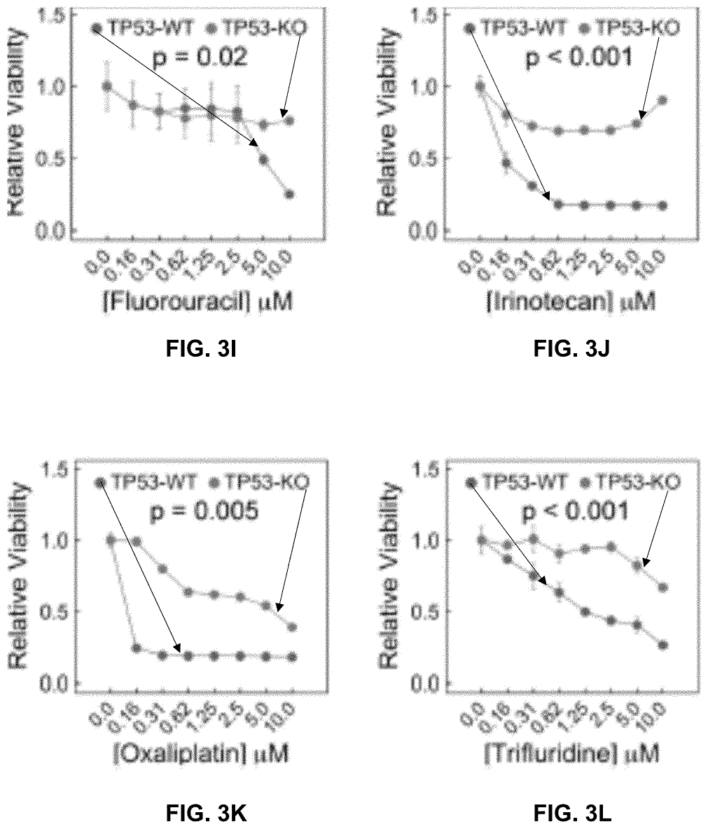

From the initial screen, ten drugs were chosen that are commonly used for the clinical management of colorectal or epithelial ovarian cancer (FIG. 3A), used Nutlin-3a as a control (FIG. 3B), and further confirmed that some of these drugs are ineffective against TP53-KO hESCs. The standard approach for epithelial ovarian chemotherapy is the combination of a platinum compound, such as Cisplatin or Carboplatin, and a taxane, such as Paclitaxel or Docetaxel. Olaparib, a PARP inhibitor, is used to treat women with advanced ovarian cancer who have BRCA1 or BRCA2 gene mutations. TP53-KO human ES cells were resistant to Cisplatin (FIG. 3G), Olaparib (FIG. 3E), and Carboplatin (FIG. 3F), whereas TP53-WT human ES cells were very sensitive to these drugs at low concentration. Both TP53 KO and WT were sensitive to Docetaxel (FIG. 3C) and Paclitaxel (FIG. 3D) at all tested concentrations.

Colorectal cancer is often treated with 5-fluorouracil, Capecitabine, Irinotecan, Oxaliplatin, and Trifluridine. Results showed that TP53-KO hESCs were resistant to Irinotecan (FIG. 3J), Oxaliplatin (FIG. 3K), and Trifluridine (FIG. 3L), whereas TP53 wild type hESCs were very sensitive to Irinotecan and Oxaliplatin at low concentration (0.16 μM), and both TP53-KO and WT hESCs were resistant to Capecitabine (FIG. 3H). Also, both TP53-KO and WT human ES cells were resistant to Fluorouracil at low concentrations (FIG. 3I). However, TP53-WT human ES cells were sensitive to Fluorouracil at high concentrations (FIG. 3I). These results suggest that a combination of both p53-dependent and p53-independent classes of drugs for chemotherapy may be more helpful to treat cancer patients.

CRISPR/Cas9 Knockout Library Screening to Resensitize p53-Null hESCs to Cisplatin.

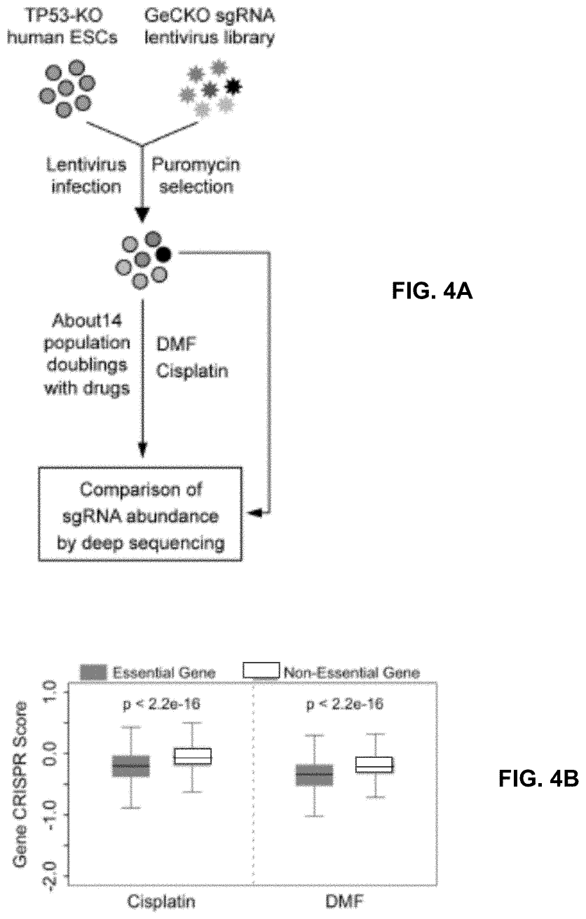

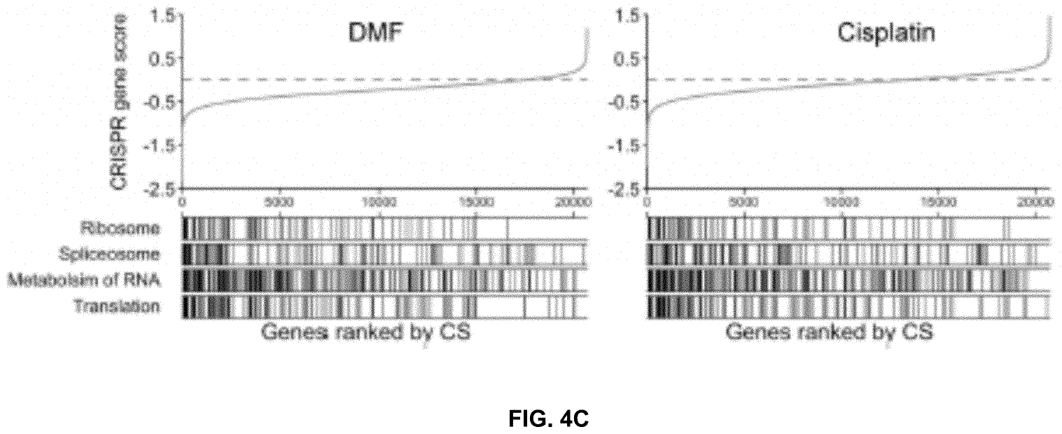

The optimal concentration of Cisplatin at which TP53-WT hESCs were very sensitive was determined, yet also at which TP53-KO hESCs were very resistant (200 nM). TP53-KO hESCs were screened in the absence and the presence of 200 nM Cisplatin to search for knockouts that would resensitize the hESCs to low concentrations of Cisplatin. Lentiviral transductions were performed at a MOI of 0.3 to make it likely that only one sgRNA virus infected per transduced cell. Sufficient cells were transduced to allow 200× coverage of each sgRNA within the library. Cells were selected for stable viral integration with puromycin for 3 days and then passaged for 14 doublings in either DMF vehicle or Cisplatin at 200 nM. At least 200× library coverage was maintained by plating >20 million cells per passage. After 14 doublings, the cells were collected and genomic DNA was extracted. Lentiviral sgRNA constructs were amplified by PCR and quantified by deep sequencing (FIG. 4A). For each human gene represented in the GECKO v2 library, its CRISPR gene score was defined as the average overall guides for a given gene of the log 2 fold changes in the abundances of each single guide sgRNAs targeting the given gene after about 14 population doublings. As a positive control for the screen performance, the CRISPR gene scores for previously published 1,580 essential genes, 927 nonessential genes, and the 2,000 non-targeting control sgRNAs that are included in the GeCKO library were evaluated. As expected, the CSs of essential and nonessential genes were significantly different (FIG. 4B), indicating that essential genes were indeed depleted during the screen, independent of the presence or the absence of Cisplatin. Gene set enrichment analysis (GSEA) was performed on all genes ranked ordered by CS, in order to agnostically group the depleted genes according to functional categories. Consistent with prior work, essential genes involved in fundamental cellular processes such as ribosome function and protein translation were strongly depleted both in control DMF (vehicle) and in Cisplatin-treated cells (FIG. 4C). These results indicate that the CRISPR screens on TP53-null hESCs with DMF or Cisplatin were valid.

### Gene Knockouts Causing Chromosome Missegregation can Resensitize TP53-Null Human ESCs to Cisplatin

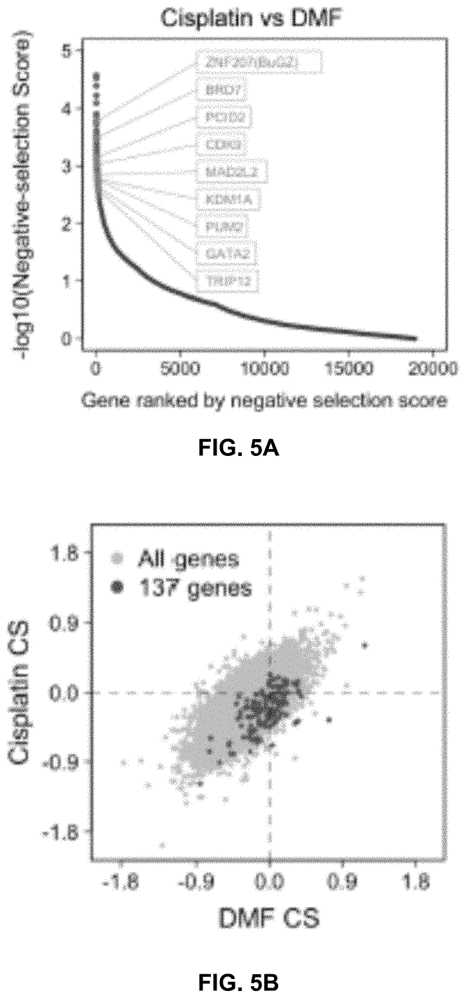

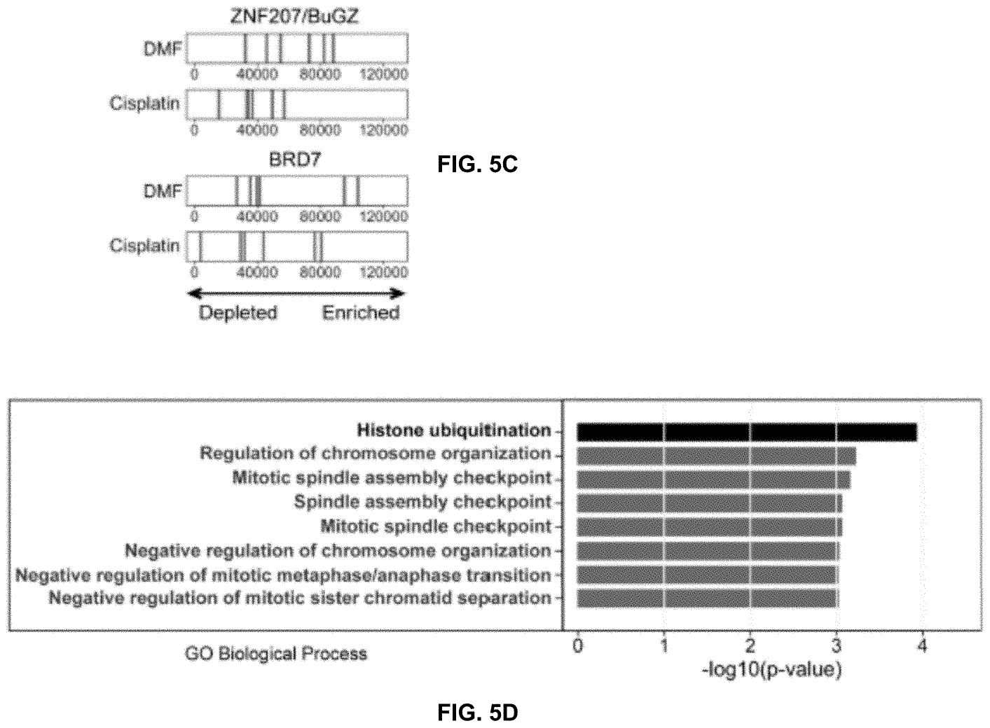

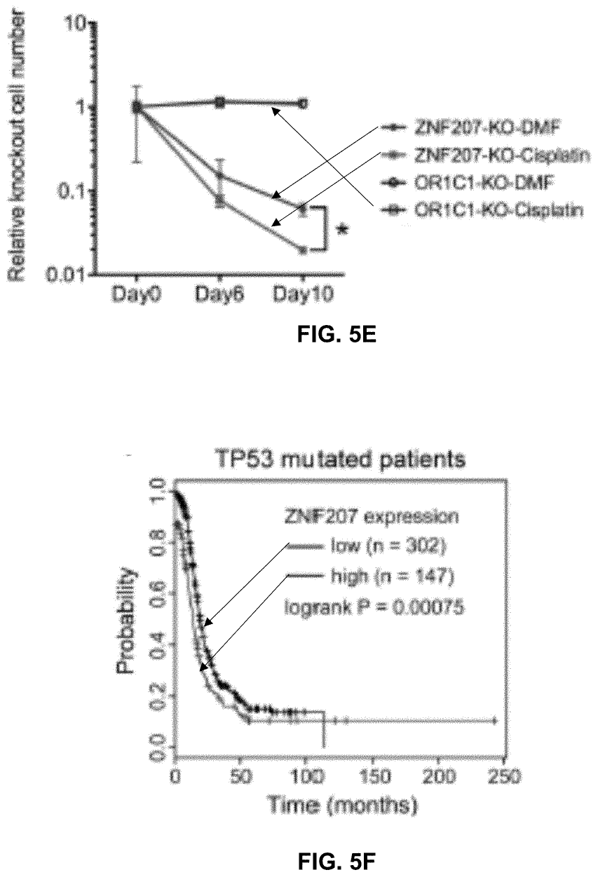

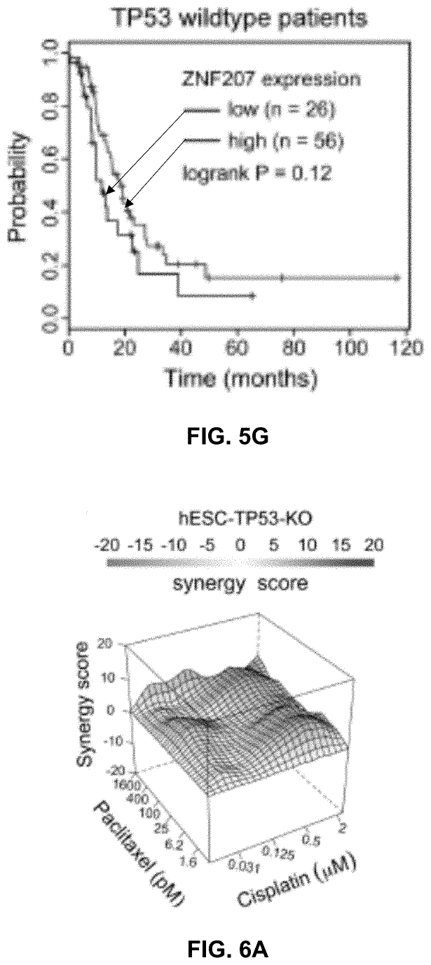

To identify genes that could resensitize TP53 knockout cells to Cisplatin, the sgRNAs counts from drug vs vehicle screens were analyzed using Model-based Analysis of Genome-wide CRISPR/Cas9 Knockout (MAGeCK) method. The MAGeCK algorithm identifies both positively and negatively selected genes simultaneously and reports robust results across different experimental conditions. MAGeCK analysis identified 137 genes significantly depleted (p<0.01) in Cisplatin treatment but not in DMF treatment (FIG. 5A). Consistent with MAGeCK analysis, inspection of the CS for these genes confirmed that all have lower CS in Cisplatin treatment than that in DMF control (FIG. 5B). The individual sgRNAs were also manually inspected and it was found that the abundances of all of the sgRNAs targeting the same gene were depleted in Cisplatin treatment compared to that in DMF control (FIG. 5C, 6 sgRNAs targeting gene ZNF207/BuGZ or BRD7 are shown). GO pathway analysis of all 137 significantly depleted genes was performed and it was found that spindle assembly checkpoint, chromosome organization, and chromatid separation genes were overrepresented among the top 20 significantly enriched GO terms (FIG. 5D and Table 1).

To functionally test one of the identified spindle assembly checkpoint genes, ZNF207/BuGZ, lentivirus was generated with two sgRNAs against gene ZNF207/BuGZ and transduced into stable Cas9-expressing TP53-KO hESC line. These cells were treated with either 200 nM Cisplatin or vehicle DMF for ten days and genomic DNA was isolated at day 6 and 10; realtime qPCR was used to quantify the ZNF207/BuGZ knockout locus, using gRNA flanking primers that only amplify when the intervening sequence has been deleted. The ZNF207/BuGZ knockout cells were depleted in both Cisplatin and control cells (consistent with it being an essential gene); however, ZNF207/BuGZ knockout cells were depleted faster in the presence of Cisplatin than in the presence of DMF vehicle control (FIG. 5E). Moreover, two more TP53-KO hESC lines were generated and the effect of Cisplatin on these two hESCs lines was validated with ZNF207/BuGZ knockout. The results showed that ZNF207/BuGZ knockout cells were more sensitive to Cisplatin. Furthermore, ZNF207 gene expression from ovarian cancer patients in published datasets with clinical outcome information was examined (patients with all stages and received chemotherapy that contained platin) and it was noted that loss of ZNF207 expression in TP53-mutated ovarian cancer predicted good response to chemotherapy containing platinum compound. However, there is no significant difference in TP53 wild type ovarian cancer (FIGS. 5F and 5G). These results suggest that genes involving chromosome missegregation are associated with Cisplatin sensitivity on TP53-null hESCs, and that pharmacological inhibitors that can be titrated may reveal a therapeutic window for sensitizing TP53 mutant cancer cells to Cisplatin. The effect of CDK9 and KDM1A gene knockout in TP53-KO hESCs in response to Cisplatin was also validated, but the difference was not significant. In addition, CDK9 and KDM1A gene expression from ovarian cancer patients in published datasets with clinical outcome information was examined. Interestingly, it was found that both loss of CDK9 and KDM1A expression predicted good responses to chemotherapy containing platinum compound in TP53-mutated ovarian cancer.

Chromosome Missegregation by Paclitaxel could Sensitize TP53-KO hESCs and TP53-KO Colon Cancer Cells to Cisplatin

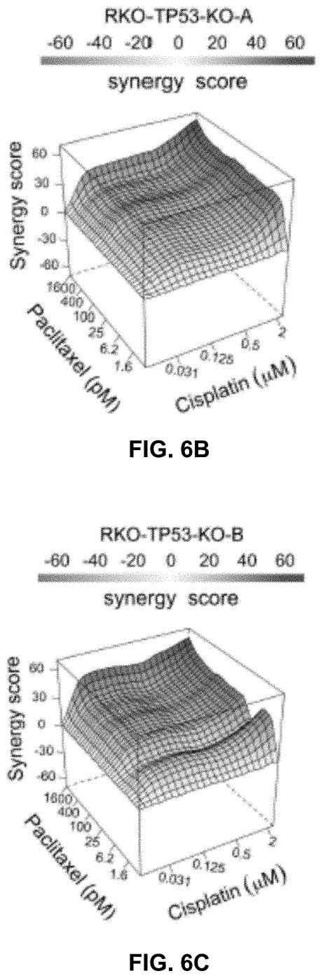

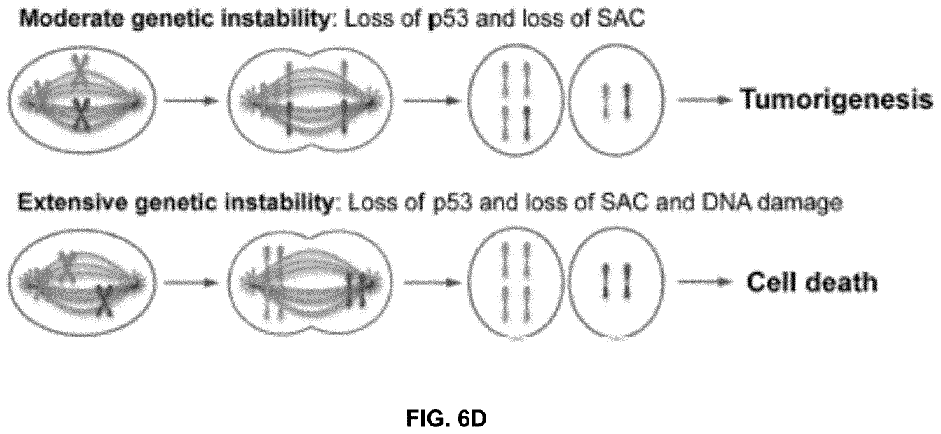

It has been reported that targeting ZNF207/BuGZ could cause chromosome misalignment due to defective interactions between microtubule and kinetochores. Paclitaxel could stabilize the microtubule polymer and protects it from disassembly lead to defects in mitotic spindle assembly, chromosome segregation, and cell division. A drug synergy experiment using low concentration of Paclitaxel and Cisplatin was performed. The inhibition of Cisplatin on TP53-KO hESCs was increased by Paclitaxel (FIG. 6A). To further validate the findings on human ESCs, two clones of TP53 knockout RKO cells were generated using CRISPR/Cas9. Like human TP53-KO ESCs, TP53-KO RKO cells are resistant to Cisplatin compared to TP53-WT RKO cells. A drug synergy experiment in human colon cancer RKO cells using Cisplatin and Paclitaxel was then performed. The inhibition of Cisplatin on TP53-KO RKO cells was increased greatly by Paclitaxel (FIGS. 6B and 6C).

TABLE 1
Top 20 GO terms of gene sets enrichment analysis.

| GO ID | Description | p-value |
|---|---|---|
| GO:0016574 | Histone ubiquitination | 0.000118963 |
| GO:0033044 | Regulation of chromosome organization | 0.000604182 |
| GO:0007094 | Mitotic spindle assembly checkpoint | 0.000707993 |
| GO:0071173 | Spindle assembly checkpoint | 0.00087609 |
| GO:0071174 | Mitotic spindle checkpoint | 0.00087609 |
| GO:2001251 | Negative regulation of chromosome | 0.000943002 |
| | organization | |
| GO:0045841 | Negative regulation of mitotic metaphase/ | 0.000968826 |
| | anaphase transition | |
| GO:2000816 | Negative regulation of mitotic sister | 0.000968826 |
| | chromatid separation | |
| GO:1902100 | Negative regulation of metaphase/ | 0.001067552 |
| | anaphase transition of cell cycle | |
| GO:1905819 | Negative regulation of chromosome | 0.001067552 |
| | separation | |
| GO:0045995 | Regulation of embryonic development | 0.001141201 |
| GO:0033048 | Negative regulation of mitotic sister | 0.001172416 |
| | chromatid segregation | |
| GO:2000177 | Regulation of neural precursor cell | 0.001396664 |
| | proliferation | |
| GO:0033046 | Negative regulation of sister chromatid | 0.001401117 |
| | segregation | |
| GO:0051985 | Negative regulation of chromosome | 0.001525227 |
| | segregation | |
| GO:0061351 | Neural precursor cell proliferation | 0.001573555 |
| GO:0031056 | Regulation of histone modification | 0.001628104 |
| GO:0031577 | Spindle checkpoint | 0.001656022 |
| GO:0010948 | Negative regulation of cell cycle process | 0.002015403 |
| GO:0051983 | Regulation of chromosome segregation | 0.002132651 |

TABLE 2
Genes displaying significant depletion (p <
0.01) in Cisplatin treatment but not in DMF treatment
as identified using MAGeCK analysis.

| | negative | negative | |
|---|---|---|---|
| | selection | selection | selection |
| id | score | p-value | rank |
| GLIS1 | 2.68E−05 | 0.00014369 | 1 |
| CNTRL | 2.99E−05 | 0.00015625 | 2 |
| EFNB2 | 4.09E−05 | 0.0002065 | 3 |
| CCDC54 | 6.05E−05 | 0.00027821 | 4 |
| RGAG4 | 8.03E−05 | 0.00036929 | 5 |
| PGLYRP4 | 0.0001245 | 0.0005771 | 6 |
| PSMC3 | 0.00013389 | 0.0006195 | 7 |
| ZNF207 | 0.00016831 | 0.00078883 | 8 |
| GLTSCR1L | 0.00018744 | 0.00087808 | 9 |
| SLC2A1 | 0.00018895 | 0.00088384 | 10 |
| NFXL1 | 0.00024099 | 0.001099 | 11 |
| ZNF230 | 0.00028191 | 0.0012937 | 12 |
| NABP1 | 0.00029 | 0.001333 | 13 |
| LIMS2 | 0.00029453 | 0.0013502 | 14 |
| BRD7 | 0.00032842 | 0.0015005 | 15 |
| ERBB3 | 0.00033399 | 0.0015313 | 16 |
| IKBKG | 0.0003411 | 0.0015664 | 17 |
| ZMAT2 | 0.00034808 | 0.0015936 | 18 |
| CNIH3 | 0.00039934 | 0.001825 | 19 |
| SYT12 | 0.00040162 | 0.0018328 | 20 |
| RPUSD4 | 0.00045516 | 0.00206 | 21 |
| THRB | 0.00050869 | 0.0022851 | 22 |
| DAOA | 0.00051895 | 0.0023333 | 23 |
| CD8A | 0.00056223 | 0.0025254 | 24 |
| PRIMA1 | 0.0005908 | 0.0026515 | 25 |
| C11orf68 | 0.00061576 | 0.0027515 | 26 |
| SETD4 | 0.00062608 | 0.0027845 | 27 |
| NR3C2 | 0.00066126 | 0.0028949 | 28 |
| ABHD16B | 0.00066929 | 0.0029253 | 29 |
| PCID2 | 0.00070303 | 0.0030399 | 30 |
| OR8G2 | 0.00072282 | 0.0031059 | 31 |
| SMARCD3 | 0.00077634 | 0.0032791 | 32 |
| TRNT1 | 0.00082986 | 0.0034608 | 33 |
| C18orf21 | 0.00084823 | 0.0035356 | 34 |
| EDC4 | 0.00086 | 0.0035738 | 35 |
| PTCHD3 | 0.00088338 | 0.0036623 | 36 |
| TRPT1 | 0.0008902 | 0.00369 | 37 |
| FUT10 | 0.00089465 | 0.0037005 | 38 |
| CDK9 | 0.00093412 | 0.0038293 | 39 |
| SCLY | 0.0009369 | 0.0038366 | 40 |
| WNT4 | 0.00099041 | 0.0040229 | 41 |
| UTF1 | 0.0010207 | 0.0041308 | 42 |
| SYT5 | 0.0010408 | 0.0042004 | 43 |
| ZNF268 | 0.0010439 | 0.0042114 | 44 |
| ORC3 | 0.0010666 | 0.0042726 | 45 |
| MAGI3 | 0.00108 | 0.0043213 | 46 |
| FCRL6 | 0.0010974 | 0.0043747 | 47 |
| FAM24A | 0.0010996 | 0.0043805 | 48 |
| CAPN7 | 0.0011202 | 0.0044548 | 49 |
| LIN54 | 0.0011509 | 0.0045548 | 50 |
| C16orf59 | 0.0011844 | 0.0046532 | 51 |
| NDUFV3 | 0.0011984 | 0.0047092 | 52 |
| SYN2 | 0.0012045 | 0.0047291 | 53 |
| GPER | 0.0012195 | 0.0047715 | 54 |
| CRYGC | 0.0012281 | 0.0047945 | 55 |
| ASTL | 0.0012479 | 0.0048704 | 56 |
| USP4 | 0.001258 | 0.0049055 | 57 |
| PVRIG | 0.0012719 | 0.0049479 | 58 |
| COL14A1 | 0.0012746 | 0.0049536 | 59 |
| ERI3 | 0.0012837 | 0.0049819 | 60 |
| KCNA5 | 0.0012958 | 0.0050175 | 61 |
| POMP | 0.0013033 | 0.0050416 | 62 |
| NKG7 | 0.0013115 | 0.0050636 | 63 |
| TMEM86A | 0.0013137 | 0.0050698 | 64 |
| TRIM3 | 0.0013508 | 0.0051845 | 65 |
| TAS2R60 | 0.001365 | 0.0052305 | 66 |
| STOML3 | 0.0014184 | 0.0054069 | 67 |
| MAD2L2 | 0.0014341 | 0.0054624 | 68 |
| TXNL1 | 0.0014461 | 0.0055085 | 69 |
| TMED1 | 0.0014533 | 0.0055279 | 70 |
| ARL14EPL | 0.0014719 | 0.0055886 | 71 |
| CCDC86 | 0.0014817 | 0.0056263 | 72 |
| GNAS | 0.00149 | 0.0056561 | 73 |
| FAM47E-STBD1 | 0.001508 | 0.002896 | 74 |
| LOC554223 | 0.0015254 | 0.0057807 | 75 |
| FAM174A | 0.0015789 | 0.0059571 | 76 |
| LRRC15 | 0.0016222 | 0.0060953 | 77 |
| MB21D2 | 0.0016324 | 0.0061324 | 78 |
| ATP2C1 | 0.0016552 | 0.0062178 | 79 |
| FAM105B | 0.001678 | 0.0062926 | 80 |
| KRT5 | 0.0016821 | 0.0063041 | 81 |
| KDM1A | 0.0016859 | 0.0063151 | 82 |
| PUM2 | 0.0017224 | 0.0064266 | 83 |
| ZNF79 | 0.0017237 | 0.0064313 | 84 |
| AKR1A1 | 0.0017517 | 0.0065292 | 85 |
| GNPNAT1 | 0.0017929 | 0.0066784 | 86 |
| LRRC27 | 0.0017994 | 0.0067046 | 87 |
| FUBP3 | 0.0018197 | 0.0067669 | 88 |
| GATAD2B | 0.0018399 | 0.0068365 | 89 |
| TBC1D21 | 0.0018427 | 0.0068443 | 90 |
| OR2L5 | 0.0018463 | 0.006859 | 91 |
| CHRFAM7A | 0.0018774 | 0.0069647 | 92 |
| TAMM41 | 0.0018804 | 0.0069757 | 93 |
| TMEM116 | 0.0018998 | 0.0070417 | 94 |
| TRAM1L1 | 0.0019533 | 0.0072296 | 95 |
| NTSR2 | 0.0019721 | 0.0072992 | 96 |
| WDR83 | 0.0020067 | 0.0074165 | 97 |
| URB2 | 0.0020179 | 0.0074494 | 98 |
| TGIF2 | 0.0020315 | 0.0075012 | 99 |
| NFKBIA | 0.0020326 | 0.0075039 | 100 |
| ZCCHC18 | 0.0020602 | 0.0075892 | 101 |
| LOH12CR1 | 0.0020927 | 0.0076939 | 102 |
| LOC100288332 | 0.0021628 | 0.0077064 | 103 |
| RAPSN | 0.0021663 | 0.007942 | 104 |
| OGN | 0.0021671 | 0.0079441 | 105 |
| CADM4 | 0.0022206 | 0.0081221 | 106 |
| PRY2 | 0.0022393 | 0.0043585 | 107 |
| NKAIN4 | 0.0022418 | 0.0081859 | 108 |
| TMCO2 | 0.0022423 | 0.008188 | 109 |
| OR51M1 | 0.0022462 | 0.0081995 | 110 |
| RIMBP2 | 0.0022704 | 0.0082814 | 111 |
| SSX5 | 0.0022741 | 0.0082943 | 112 |
| GATA2 | 0.002289 | 0.0083435 | 113 |
| PRR11 | 0.0023275 | 0.0084654 | 114 |
| SLFN5 | 0.002381 | 0.0086445 | 115 |
| DYRK2 | 0.0023828 | 0.0086476 | 116 |
| MLL2 | 0.0023878 | 0.0067103 | 117 |
| DCAF10 | 0.0024344 | 0.0088334 | 118 |
| GLRA1 | 0.0024842 | 0.0090062 | 119 |
| SLC38A10 | 0.0024879 | 0.0090145 | 120 |
| BDKRB1 | 0.002493 | 0.0090329 | 121 |
| TRIP12 | 0.0025413 | 0.0091915 | 122 |
| POLR2C | 0.0025861 | 0.009359 | 123 |
| HAGH | 0.0026167 | 0.0094537 | 124 |
| LGR6 | 0.0026482 | 0.0095647 | 125 |
| RCC1 | 0.0026544 | 0.0095851 | 126 |
| SPATA31A2 | 0.0026574 | 0.0077467 | 127 |
| CSN3 | 0.002675 | 0.0096531 | 128 |
| TRIM42 | 0.0026785 | 0.0096636 | 129 |
| TAF1C | 0.0027016 | 0.0097458 | 130 |
| PCGF1 | 0.0027082 | 0.0097709 | 131 |
| ATN1 | 0.0027369 | 0.009862 | 132 |
| SLC25A34 | 0.0027479 | 0.009895 | 133 |
| POGLUT1 | 0.0027551 | 0.009917 | 134 |
| MPP1 | 0.0027671 | 0.0099557 | 135 |
| HMX1 | 0.0027809 | 0.0099991 | 136 |
| SST | 0.0028041 | 0.010076 | 137 |

What is claimed is:

1. A method for treating a patient having been diagnosed with a cancer having a TP53 mutation comprising:
delivering an inhibitor to the patient via an administration route,
wherein the inhibitor targets a gene associated with spindle cell assembly checkpoint regulation or a protein expressed from a gene associated with spindle cell assembly checkpoint regulation.

2. The method of claim 1, wherein the gene associated with spindle cell assembly checkpoint regulation includes one or more of ZNF207, BRD7, PCID2, CDK9, MAD2L2, KDM1A, PUM2, GATA2, and TRIP12, and wherein the protein expressed from the gene associated with spindle cell assembly checkpoint regulation corresponds to a translation product of one or more of ZNF207, BRD7, PCID2, CDK9, MAD2L2, KDM1A, PUM2, GATA2, and TRIP12.

3. The method of claim 2, wherein the inhibitor comprises a binding region of an antibody, and wherein the binding region targets an epitope of the protein.

4. The method of claim 2, wherein the inhibitor comprises a miRNA comprising a substantially complementary sequence to a RNA product of the gene.

5. The method of claim 4, wherein delivering the inhibitor comprising the miRNA comprises:
delivering a vector including heterologous DNA expressing the miRNA.

6. The method of claim 2, further comprising delivering a vector including heterologous DNA expressing one or more sgRNAs.

7. The method of claim 6, wherein the vector includes a lentivirus.

8. The method of claim 1, further comprising: delivering a therapeutic agent.

9. The method of claim 8, wherein the therapeutic agent comprises one or more from the group: Bendamustine hydrochloride, Bleomycin sulfate, Carboplatin, Carmustine, Chlorambucil, Cisplatin, Cladribine, Clofarabine, Cytarabine hydrochloride, Decitabine, Dexrazoxane, Estramustine phosphate sodium, Etoposide, Irinotecan hydrochloride, Melphalan hydrochloride, Mitomycin, Olaparib, Osimertinib, Oxaliplatin, Pipobroman, Teniposide, Thiotepa, Topotecan hydrochloride, Triethylenemelamine, Trifluridine, Uracil mustard, and Valrubicin.

10. A method for selecting a treatment course for a patient having been diagnosed with a cancer comprising:
determining a genetic profile for the cancer, the genetic profile comprising a TP53 gene sequence;
comparing the TP53 gene sequence to a native TP53 gene sequence;
selecting, based at least in part on the comparison, the treatment course, the treatment course not including a resistant drug.

11. The method of claim 10, wherein the genetic profile demonstrates a mutation to the TP53 gene compared to the native TP53 gene, and wherein the resistant drug includes one or more of the group: Bendamustine hydrochloride, Bleomycin sulfate, Carboplatin, Carmustine, Chlorambucil, Cisplatin, Cladribine, Clofarabine, Cytarabine hydrochloride, Decitabine, Dexrazoxane, Estramustine phosphate sodium, Etoposide, Irinotecan hydrochloride, Melphalan hydrochloride, Mitomycin, Olaparib, Osimertinib, Oxaliplatin, Pipobroman, Teniposide, Thiotepa, Topotecan hydrochloride, Triethylenemelamine, Trifluridine, Uracil mustard, and Valrubicin.

12. The method of claim 11, wherein the mutation comprises a deletion of at least a portion of the TP53 gene sequence.

13. The method of claim 12, wherein the deletion comprises all of the TP53 gene sequence.

14. The method of claim 10, further comprising obtaining a biopsy of the cancer, and wherein determining the genetic profile comprises:
sequencing DNA from the biopsy of the cancer to determine a genetic sequence for at least one allele encoding the TP53 gene.

15. A method for treating a patient having been diagnosed with a cancer having a TP53 mutation comprising:
delivering an inhibitor to the patient via an administration route,
wherein the inhibitor targets at least one or the genes or a translation product of said genes from the group: GLIS1, CNTRL, EFNB2, CCDC54, RGAG4, PGLYRP4, PSMC3, ZNF207, GLTSCR1L, SLC2A1, NFXL1, ZNF230, NABP1, LIMS2, BRD7, ERBB3, IKBKG, ZMAT2, CNIH3, SYT12, RPUSD4, THRB, DAOA, CD8A, PRIMA1, C11orf68, SETD4, NR3C2, ABHD16B, PCID2, OR8G2, SMARCD3, TRNT1, C18orf21, EDC4, PTCHD3, TRPT1, FUT10, CDK9, SCLY, WNT4, UTF1, SYTS, ZNF268, ORC3, MAGI3, FCRL6, FAM24A, CAPN7, LIN54, C16orf59, NDUFV3, SYN2, GPER, CRYGC, ASTL, USP4, PVRIG, COL14A1, ERI3, KCNAS, POMP, NKG7, TMEM86A, TRIM3, TAS2R60, STOML3, MAD2L2, TXNL1, TMED1, ARL14EPL, CCDC86, GNAS, FAM47E-STBD1, L00554223, FAM174A, LRRC15, MB21D2, ATP2C1, FAM105B, KRTS, KDM1A, PUM2, ZNF79, AKR1A1, GNPNAT1, LRRC27, FUBP3, GATAD2B, TBC1D21, OR2L5, CHRFAM7A, TAMM41, TMEM116, TRAM1L1, NTSR2, WDR83, URB2, TGIF2, NFKBIA, ZCCHC18, LOH12CR1, LOC100288332, RAPSN, OGN, CADM4, PRY2, NKAIN4, TMCO2, OR51M1, RIMBP2, SSXS, GATA2, PRR11, SLFNS, DYRK2, MLL2, DCAF10, GLRA1, SLC38A10, BDKRB1, TRIP12, POLR2C, HAGH, LGR6, RCC1, SPATA31A2, CSN3, TRIM42, TAF1C, PCGF1, ATN1, SLC25A34, POGLUT1, MPP1, HMX1 and SST, and
wherein the inhibitor acts to reduce expression of the gene, reduce function of the protein, or both.


SEQUENCE LISTING (13 sequences)

SEQ ID NO: 1 - DNA, 20 bp, Artificial Sequence
  Description of Artificial Sequence Synthetic oligonucleotide
ccattgttcaatatcgtccg

SEQ ID NO: 2 - DNA, 20 bp, Artificial Sequence
  Description of Artificial Sequence Synthetic oligonucleotide
gggcagctacggtttccgtc

SEQ ID NO: 3 - DNA, 25 bp, Artificial Sequence
  Description of Artificial Sequence Synthetic primer
tgtacaaaaaagcaggctttaaagg

SEQ ID NO: 4 - DNA, 25 bp, Artificial Sequence
  Description of Artificial Sequence Synthetic primer
taatgccaactttgtacaagaaagc

SEQ ID NO: 5 - DNA, 87 bp, Artificial Sequence
  Description of Artificial Sequence Synthetic primer
  modified_base (59)..(63): a, c, t, g, unknown or other
aatgatacggcgaccaccgagatctacactctttccctacacgacgctcttccgatctnnnnntcttgtggaaaggacgaaacaccg

SEQ ID NO: 6 - DNA, 84 bp, Artificial Sequence
  Description of Artificial Sequence Synthetic primer
  modified_base (25)..(30): a, c, t, g, unknown or other
caagcagaagacggcatacgagatnnnnnngtgactggagttcagacgtgtgctcttccgatcttgtgggcgatgtgcgctctg

SEQ ID NO: 7 - DNA, 33 bp, Artificial Sequence
  Description of Artificial Sequence Synthetic primer
acactctttccctacacgacgctcttccgatct

SEQ ID NO: 8 - DNA, 20 bp, Artificial Sequence
  Description of Artificial Sequence Synthetic primer
ggttgggaaagtgagggatt

SEQ ID NO: 9 - DNA, 22 bp, Artificial Sequence
  Description of Artificial Sequence Synthetic primer
aacacttctcacaggaacttgc

SEQ ID NO: 10 - DNA, 20 bp, Artificial Sequence
  Description of Artificial Sequence Synthetic primer
cagcctccttctgtgtgtga

SEQ ID NO: 11 - DNA, 20 bp, Artificial Sequence
  Description of Artificial Sequence Synthetic primer
tgcttgccctgagtagaggt

SEQ ID NO: 12 - DNA, 20 bp, Artificial Sequence
  Description of Artificial Sequence Synthetic primer
aaagccgctcaactacatgg

SEQ ID NO: 13 - DNA, 23 bp, Artificial Sequence
  Description of Artificial Sequence Synthetic primer
ctctatttccttcagttctgctc